(19) United States
(12) Patent Application Publication
(10) Pub. No.: US 2026/0265782 A1
(43) Pub. Date: Sep. 10, 2026

(54) EDITABLE CELL LINES

(71) Applicant: LONZA SALES AG, Basel (CH)

(72) Inventors: Ariel GILERT, Basel (CH); Katarina RADOSEVIC, Basel (CH); Bracha SHRAIBMAN, Basel (CH); Frida Grynspan GOTLIEB, Basel (CH); Marc FEARY, Basel (CH); Bernadette SWEENEY, Basel (CH)

(21) Appl. No.: 18/876,561

(22) PCT Filed: Jun. 29, 2023
(86) PCT No.: PCT/US2023/069409

§ 371 Date: Dec. 18, 2024

Related U.S. Application Data
(60) Provisional application No. 63/367,413, filed on Jun. 30, 2022.

(51) Int. Cl. C12N 15/90; C07K 16/00; C12N 9/22; C12N 15/11

(52) U.S. Cl. CPC C12N 15/907; C07K 16/00; C12N 9/226; C12N 15/11; C12N 2310/20

(57) ABSTRACT

The present disclosure provides editable cell lines, including the use of gene editing proteins to produce the cell lines. The editable cell lines are able to express antibody constant regions that can serve as a platform for the antibody variable regions to produce customized antibody.

### FIELD OF THE INVENTION

The present disclosure provides editable cell lines, including the use of gene editing proteins to produce the cell lines. By preparing editable cell lines that contain the ability to be further modified to individually produce a desired antibody, the cost and time for antibody manufacturing process can be reduced.

### BACKGROUND OF THE INVENTION

As clinical adoption of advanced antibody therapies begins to gain traction, more attention is turning to the underlying manufacturing strategies that will allow these therapies to benefit patients worldwide. While antibody therapies hold great promise clinically, high manufacturing costs relative to reimbursement present a formidable roadblock to commercialization.

One of the challenges facing antibody therapies is the multi-staged and complex manufacturing process to produce the desired antibodies. Current manufacturing processes rely on introducing vectors to construct cell lines to express the gene of interest and to obtain the desired antibodies. This process requires vector construction for each new antibody to be expressed followed by gene introduction and pool recovery. The process further requires clone selection to find high producing clones, which is time consuming with each clone requiring stability assessment and introduces the potential for deviations and failure.

What are needed to overcome these challenges are methods to shorten and simplify the manufacturing process. Editable cell lines are able to express antibody constant regions and can serve as a platform for the antibody variable regions and remove the need for vector construction and provide a reliable antibody manufacturing platform. The present invention fulfills these needs.

### SUMMARY OF THE INVENTION

In some embodiments, the disclosure provides a cell, comprising: a genomic nucleic acid sequence comprising, a first sequence encoding antibody heavy chain constant regions 1, 2 and 3 on one strand of the genomic nucleic acid sequence, wherein the first sequence is not flanked by a sequence encoding an antibody heavy chain variable region, and a second sequence encoding antibody light chain constant region 1 on an opposite strand of the genomic nucleic acid sequence, wherein the second sequence is not flanked by a sequence encoding an antibody light chain variable region.

In further embodiments, provided is a method of producing an editable cell comprising: introducing a first sequence encoding antibody heavy chain constant regions 1, 2 and 3 on one strand of a genomic nucleic acid sequence of a cell, and introducing a second sequence encoding antibody light chain constant region 1 on an opposite strand of the genomic nucleic acid sequence of the cell.

Also provided herein is a method of making an antibody producing editable cell, comprising: providing a cell comprising, a genomic nucleic acid sequence comprising, a first sequence encoding antibody heavy chain constant regions 1, 2 and 3 on one strand of the genomic nucleic acid sequence, wherein the first sequence is not flanked by a sequence encoding an antibody heavy chain variable region, and a second sequence encoding antibody light chain constant region 1 on an opposite strand of the genomic nucleic acid sequence, wherein the second sequence is not flanked by a sequence encoding an antibody light chain variable region, introducing a sequence encoding an antibody heavy chain variable region to the first sequence upstream of the antibody heavy chain constant regions, and introducing a sequence encoding an antibody light chain variable region to the second sequence upstream of the antibody light chain constant region 1.

### BRIEF DESCRIPTION OF THE DRAWINGS

FIG. 1 shows schematic representation of how an intermediate headless antibody can be modified with the introduction of antibody variable regions to create a desired customized antibody.

FIG. 2 shows an exemplary representation of genomic nucleic acid sequence of an editable cell wherein the antibody heavy chain constant region and light chain constant region are located on the opposite strands of the genomic sequence within the same locus.

FIG. 3 shows an exemplary representation of genomic nucleic acid sequence wherein the editable cell line is modified to include the sequence encoding antibody heavy constant region and the sequence encoding antibody light constant region on the opposite strands of the genomic sequence within the same locus as well as the sequences encoding the gene editing protein, selection marker, and an inducible promoter.

FIGS. 4A-4C show schematic representation of de-novo headless cell line (4A), cells' recovery following transfection (4B), as well as FACS analysis showing the integration of the cassette into BOB cells (GFP expressing CHO cells) using the SSI system (4C).

FIGS. 5A-5C show PCR validation of generation of headless antibody (5A) and full antibody (5B and 5C).

FIGS. 6A-6B show antibody expression validation by WB (6A) and functionality and expression validation by FACS analysis (6B).

FIG. 7 shows schematic representation of seamless integration of heavy chain and light chain variable regions using one DNA template and selection marker.

FIG. 8 shows cells recovery following Puromycin antibiotic selection of cells transfected with heavy chain and light chain variable regions.

FIG. 9A-9B show antibody functionality and expression validation by FACS analysis (9A) and antibody expression validation by WB (9B).

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, cells, and/or nucleic acids of the invention can be used to achieve any of the methods as described herein.

As described throughout, the subject of this disclosure is an editable cell line that is in embodiments, stable, and in further embodiments, high producing, capable of further modification to individually produce a desired, customized antibody. The editable cell line includes the use of gene editing proteins to further modify the genomic sequence encoding the headless antibody structure. The editable cell line may or may not express the headless antibody structure because the headless antibody structure is an intermediate. However, once the editable cell line is fully modified with antibody variable regions, the cell line can be expressed to produce fully customized antibody protein.

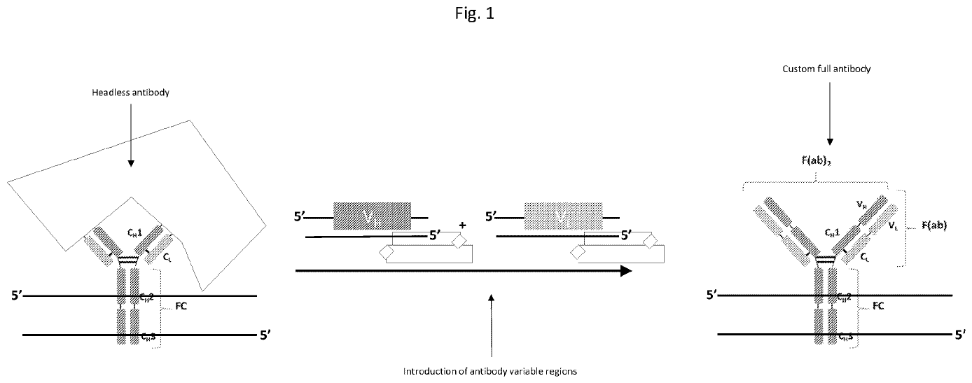

As used herein, “headless antibody” means an antibody protein without the antibody variable regions, but that includes constant heavy chain and light chain regions. The antibody variable regions include heavy chain and light chain variable regions that define the antigen binding site of the antibody protein. See, e.g., FIG. 1, showing a representation of an antibody that includes the heavy chain and light chain constant regions. The headless antibody structure is capable of modification and is only an intermediate, wherein antibody variable regions can be introduced to produce customized antibody proteins.

As used herein, “genomic nucleic acid” or “genomic sequence” means nucleic acids that are integrated into the genome of the cell. The term “genome” refers to the complete set of genetic information in the chromosomes of the cells.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, gRNA, mRNA, tRNA, rRNA, snRNA, microRNA, miRNA, or MIRNA.

A “gene” as used herein refers to an assembly of nucleotides that encode a polypeptide and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

As used herein, “stable” means the cell line can maintain cell integrity with the use of commonly used storage methods and the cells are capable of maintaining antibody producing function over the course of multiple cell divisions. In embodiments, the cell lines described herein are stable cell lines. As used herein, “high producing” or “high expressing” means producing the molecule of interest in the amount of at least about 1 g/L. The amount that is considered high producing will depend on the molecule of interest being produced and may be about 2 g/L, about 3 g/L, about 4 g/L, about 5 g/L, about 6 g/L, about 7 g/L, about 8 g/L, about 9 g/L, about 10 g/L, about 15 g/L, about 20 g/L or higher. In embodiments, the cells provided herein are stable, high expressing cells.

FIG. 1 shows a schematic representation of how an intermediate headless antibody can be modified with the introduction of antibody variable regions to create a desired customized antibody. In FIG. 1, the overlapping antibody structure represents what is encoded and will be produced by expressing the DNA sequence. Accordingly, initially the sequence represents a headless antibody. Antibody variable regions are then introduced to the genome. Finally, the full structure of the customized antibody is expressed. Various methods for producing such headless antibody structures are described herein.

An editable cell line of this disclosure suitably does not require the use of DNA vectors to introduce the genetic sequence encoding the antibody for each antibody production. One commonly used method of producing antibody requires the use of recombinant DNA vectors, which are created, cloned, and introduced into the host cells for each antibody production cycle. However, this general method depends on the random/semi-random integration of the DNA vectors into the host cells.

An editable cell line can reduce the time and cost of the commonly used antibody production method by removing the need to produce DNA vectors, introducing the vectors to the host cells, and selecting the host cells with DNA vectors present to produce the antibodies. In the disclosed editable cell lines, the sequence encoding the headless antibody is integrated into the genomic sequence of the host cell. Therefore, once the editable cells are modified to produce the full antibody, said cells can be selected and cloned to produce antibodies.

An editable cell line can be produced by integrating the sequence encoding antibody constant regions into the genomic sequence of the cell and select for the suitably stable, and in embodiments, high expressing cell line. The resulting cell contains the sequence encoding the headless antibody.

In some embodiments, provided herein is a method of producing an editable cell, comprising: introducing a first sequence encoding antibody heavy chain constant regions 1, 2, and 3 on one strand of a genomic nucleic acid sequence of a cell, and introducing a second sequence encoding antibody light chain constant region 1 on an opposite strand of the genomic nucleic acid sequence of the cell. In some embodiments, the first and the second sequences are located on the opposite strands of the genomic nucleic acid within the same locus. In some embodiments, the first and the second sequences are on the opposite strands within the same locus are separated by 1, 2, 3, 4, 5, 6, 7, 8, 9, or 10 nucleotide base pairs. In some embodiments, the first and the second sequences are located on different chromosomes.

In some embodiments, provided herein is a method of producing an editable cell, further comprising: introducing a sequence encoding the gene editing protein to the genomic nucleic acid sequence and expressing the gene editing protein. In some embodiments, provided herein is a method of producing an editable cell, further comprising: introducing a ribonucleoprotein (RNP) of the suitable gene editing protein. In some embodiments, provided herein is a method of producing an editable cell, further comprising: introducing a plasmid containing the sequence encoding the gene editing protein to the cell and expressing the gene editing protein sequence in the plasmid.

As used herein, the terms “engineered nuclease”, “engineered gene editing protein”, or “gene editing protein” refer to a nuclease that has been separated, modified, mutated, and/or altered from its natural state as a nuclease. A “nuclease” refers to an enzyme that is able to cut a DNA and/or RNA molecule. By engineering the nuclease, the specific location of the cut can be designed and tailored to the desired cell type and/or gene of interest.

Exemplary engineered nucleases that can be inserted into the cell (either produced from integrated, genomic nucleic acids, viral or other non-genomic nucleic acids, or as RNP) include, for example, a meganuclease, a methyltransferase a zinc finger nuclease, a transcription activator-like effector-based nuclease (TALENS), a FokI nuclease, and a CRISPR-associated (Cas) nuclease. In general, engineered nucleases use a DNA-binding protein which has both a desired catalytic activity and the ability to bind the desired target sequence through a protein-nucleic-acid interaction in a manner similar to restriction enzymes. Examples include meganucleases which are naturally occurring or engineered rare sequence cutting enzymes, zinc finger nucleases (ZFNs) or transcription activator-like nucleases (TALENs) which contain the FokI catalytic nuclease subunit linked to a modified DNA binding domain and can cut one predetermined sequence each. In ZFNs the binding domain is comprised of chains of amino-acids folding into customized zinc finger domains. In TALENs, similarly, 34 amino acid repeats originating from transcription factors fold into a huge DNA-binding domain. In the event of gene targeting, these enzymes can cleave genomic DNA to form a double strand break (DSB) or create a nick which can be repaired by one of two repair pathways, non-homologous end joining (NHEJ) or homologous recombination (HR). The NHEJ pathway can potentially result in specific mutations, deletions, insertions or replacement events. The HR pathway results in replacement of the targeted sequence by a supplied donor sequence. Exemplary FokI and methyltransferase-based systems are described in U.S. Pat. No. 10,220,052, the disclosure of which is incorporated by reference herein in its entirety.

The Clustered Regularly Interspaced Short Palindromic Repeats (CRISPR) and associated proteins (CRISPR-associated nucleases, or Cas proteins), which comprise the CRISPR-Cas system, were first identified in selected bacterial species and form part of a prokaryotic adaptive immune system. See Sorek, et al., “CRISPR—a widespread system that provides acquired resistance against phages in bacteria and archaea,” Nat. Rev. Microbiol. 6(3)181-6 (2008), which is incorporated by reference herein in its entirety. CRISPR-Cas systems have been classified into three main types: Type I, Type II, and Type III. The main defining features of the separate Types are the various cas genes, and the respective proteins they encode, that are employed. The cas1 and cas2 genes appear to be universal across the three main Types, whereas cas3, cas9, and cas10 are thought to be specific to the Type I, Type II, and Type III systems, respectively. See, e.g., Barrangou, R. and Marraffini, L. A., “CRISPR-Cas systems: prokaryotes upgrade to adaptive immunity,” Mol. Cell. 54(2):234-44 (2014), which is incorporated by reference herein in its entirety.

In general, the CRISPR-Cas system functions by capturing short regions of invading viral or plasmid DNA and integrating the captured DNA into the host genome to form so-called CRISPR arrays that are interspaced by repeated sequences within the CRISPR locus. This acquisition of DNA into CRISPR arrays is followed by transcription and RNA processing.

Depending on the bacterial species, CRISPR RNA processing proceeds differently. For example, in the Type II system, originally described in the bacterium Streptococcus pyogenes, the transcribed RNA is paired with a transactivating RNA (tracrRNA) before being cleaved by RNase III to form an individual CRISPR-RNA (crRNA). The crRNA is further processed after binding by the Cas9 nuclease to produce the mature crRNA. The crRNA/Cas9 complex subsequently binds to DNA containing sequences complimentary to the captured regions (termed protospacers). The Cas9 protein then cleaves both strands of DNA in a site-specific manner, forming a double-strand break (DSB). This provides a DNA-based memory, resulting in rapid degradation of viral or plasmid DNA upon repeat exposure and/or infection.

Since its original discovery, multiple groups have done extensive research around potential applications of the CRISPR system in genetic engineering, including gene editing (Jinek et al., “A programmable dual-RNA-guided DNA endonuclease in adaptive bacterial immunity,” Science 337(6096):816-21 (2012); Cong et al., “Multiplex genome engineering using CRISPR/Cas systems,” Science 339(6121):819-23 (2013); and Mali et al., “RNA-guided human genome engineering via Cas9,” Science 339(6121):823-26; each of which is incorporated by reference herein in its entirety). One major development was utilization of a chimeric RNA to target the Cas9 protein, designed around individual units from the CRISPR array fused to the tracrRNA. This creates a single RNA species, called a small guide RNA (gRNA) where modification of the sequence in the protospacer region can target the Cas9 protein site-specifically. Considerable work has been done to understand the nature of the base-pairing interaction between the chimeric RNA and the target site, and its tolerance to mismatches, which is highly relevant in order to predict and assess off-target effects (see, e.g., Fu et al., “Improving CRISPR-Cas nucleases using truncated guide RNAs,” Nature Biotechnology 32(3):279-84 (2014), and supporting material, which is incorporated by reference herein in its entirety).

The CRISPR-Cas9 gene editing system has been used successfully in a wide range of organisms and cell lines, both in order to induce double-strand break formation using the wild type Cas9 protein or to nick a single DNA strand using a mutant protein termed Cas9n/Cas9 D10A (see, e.g., Mali et al., (2013) and Sander and Joung, “CRISPR-Cas systems for editing, regulating and targeting genomes,” Nature Biotechnology 32(4):347-55 (2014), each of which is incorporated by reference herein in its entirety). While double-strand break (DSB) formation results in creation of small insertions and deletions (indels) that can disrupt gene function, Cas9 wild-type as well as Cas9n/Cas9 D10A nickase can avoid indel creation (the result of repair through non-homologous end-joining) while stimulating the endogenous homologous recombination machinery. Thus, these systems can be used to insert regions of DNA into the genome with high-fidelity.

In some embodiments, provided herein is a method of producing an editable cell, wherein the gene editing protein utilized in the methods, is a CRISPR-associated gene editing protein. In suitable embodiments, the CRISPR-associated (Cas) nuclease is a Cas9 nuclease, or can be other Cas nucleases such as Cas12, Cas12i2, Cas13, Cas14, MAD7 (Cas12a), etc. In some embodiments, the Cas9 nuclease is a Cas9 nuclease that has reduced immunogenicity, such as disclosed in U.S. Published Patent Application No. 2018-0319850, the disclosure of which is incorporated by reference herein in its entirety. In some embodiments, the gene editing protein is a zinc finger nuclease. In some embodiments, the gene editing protein is a TALENS. In some embodiments, the gene editing protein is a FokI nuclease.

In addition to a Cas9 nuclease, Cas12, Cas13, Cas14, and MAD7 (Cas12a) nucleases can also be utilized in the methods described herein. Cas12 creates staggered cuts in dsDNA (5 nucleotide 5′ overhang dsDNA break). Cas12 processes its own guide RNAs, leading to increased multiplexing ability. Cas13t targets RNA, not DNA. Once it is activated by a ssRNA sequence bearing complementarity to its crRNA spacer, it unleashes a nonspecific RNase activity and destroys all nearby RNA regardless of their sequence. See, e.g., Yan et al., “CRISPR-Cas12 and Cas13: the lesser known siblings of CRISPR Cas9,” Cell Biology and Toxicology pages 1-4 (Aug. 29, 2019), the disclosure of which is incorporated by reference herein in its entirety. In some embodiments, the Cas12i2 nuclease can also be utilized in the methods described herein, such as disclosed in U.S. Pat. No. 10,808,245, the disclosure of which is incorporated by reference herein in its entirety.

In some embodiments, provided herein is a method of producing an editable cell, wherein the sequence encoding the gene editing protein (genomically integrated) is operably connected to an inducible promoter. By placing the gene editing protein under the control of an inducible promoter, the nuclease can be kept dormant or silent prior to its desired use as a gene editing tool. In some embodiments, the inducible promoter is a TET-on system.

As used herein, a “promoter,” “promoter sequence,” or “promoter region,” which refers to a DNA regulatory region/sequence capable of binding RNA polymerase and initiating transcription of a downstream coding or non-coding gene sequence. In other words, the promoter and the gene are in operable combination or operably linked. As referred to herein, the terms “in operable combination”, “in operable order”, “operably connected”, and “operably linked” refer to the linkage of nucleic acid sequences in such a manner that a promoter capable of directing the transcription of a given gene and/or the synthesis of a desired protein molecule is produced. The term also refers to the linkage of amino acid sequences in such a manner so that a protein is produced.

In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

Various promoters may be used to drive the gene expression. In some embodiments, the promoter is an “inducible promoter”, i.e., the promoter is not constitutively expressing any of the gene products as described herein and is activated in response to specific stimuli that can be turned on or off, depending on the desired control of the gene that is under control of the promoter. In other embodiments as described herein, the promoter is a constitutive promoter, which initiates mRNA synthesis independent of the influence of an external regulation.

Suitably, the promoters used to control the engineered nucleases are derepressible promoters. As used herein, a “derepressible promoter” refers to a structure that includes a functional promoter and additional elements or sequences capable of binding to a repressor element to cause repression of the functional promoter. “Repression” refers to the decrease or inhibition of the initiation of transcription of a downstream coding or non-coding gene sequence by a promoter. A “repressor element” refers to a protein or polypeptide that is capable of binding to a promoter (or near a promoter) so as to decrease or inhibit the activity of the promoter. A repressor element can interact with a substrate or binding partner of the repressor element, such that the repressor element undergoes a conformation change. This conformation change in the repressor element takes away the ability of the repressor element to decrease or inhibit the promoter, resulting in the “derepression” of the promoter, thereby allowing the promoter to proceed with the initiation of transcription. A “functional promoter” refers to a promoter, that absent the action of the repressor element, would be capable of initiation transcription. Various functional promoters that can be used in the practice of the present invention are known in the art, and include for example, PCMV, PH1, P19, P5, P40 and promoters of Adenovirus helper genes (e.g., E1A, E1B, E2A, E4Orf6, and VA).

Examples of various controllable promoters, including inducible promoters and derepressible promotors are described herein, as are methods of inducing expression of the Cas9 nuclease via the introduction of a molecule that induces expression, or that derepresses a derepressible promoter.

Exemplary repressor elements and their corresponding binding partners that can be used as derepressible promoters are known in the art, and include systems such as the cumate gene-switch system (CuO operator, CymR repressor and cumate binding partner) (see, e.g., Mullick et al., “The cumate gene-switch: a system for regulated expression in mammalian cells,” BMC Biotechnology 6:43 (1-18) (2006), the disclosure of which is incorporated by reference herein in its entirety, including the disclosure of the derepressible promoter system described therein) and the TetO/TetR system described herein (see, e.g., Yao et al., “Tetracycline Repressor, tetR, rather than the tetR-Mammalian Cell Transcription Factor Fusion Derivatives, Regulates Inducible Gene Expression in Mammalian Cells,” Human Gene Therapy 9:1939-1950 (1998), the disclosure of which is incorporated by reference herein in its entirety). In exemplary embodiments, the derepressible promoters comprise a functional promoter and either one two tetracycline operator sequences (TetO or TetO₂). In such embodiments, the nucleic acid introduced into the T-cells further includes a tetracycline repressor protein to control the TetO derepressible system (a TET-on system).

As described herein, the methods can further include inducing expression of the CRISPR-associated nuclease by activating the inducible promoter. In the case of an inducible promoter, such as a 4HT inducible promoter, a rapamycin inducible promoter, a hormone response element, or a glutamate inducible promoter, the promoter is induced by the addition of, for example, 4-hydroxytamoxifen, rapamycin, a hormone, or glutamate, respectively. In the case of a derepressible promoter, such as the TetO sequence described herein coupled to a CMV promoter, the addition of doxycycline removes the repression, and allows the gene (engineered nuclease) to be expressed via the CMV promoter. Suitably, the nucleic acid molecule that encodes the Cas9 also encodes a TetR repressor element, suitably under the control of another promoter system, such as a constitutive promoter like the hPGK promoter.

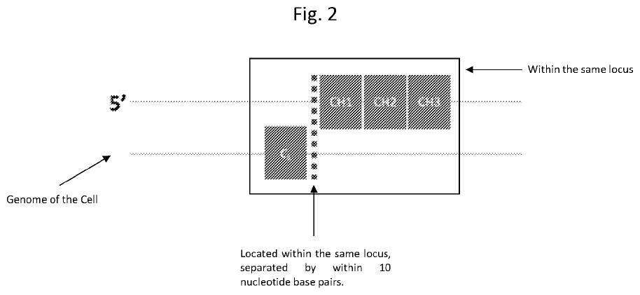

FIG. 2 shows representation of the method of producing an editable cell by introducing a first sequence encoding antibody heavy chain constant regions 1, 2, and 3, represented by CH1, CH2, and CH3, respectively, and introducing a second sequence encoding antibody light chain constant region 1, to the genomic nucleic acid sequence, represented by CL. In FIG. 2, the first and the second sequences are located on the opposite strands of the genomic nucleic acid but are within the same locus and are separated by 10 or less nucleotide base pair. As described herein, other orientations, including situations in which the antibody heavy chain constant regions are on a first nucleic acid sequence of a completely different chromosome as the second sequence encoding the antibody light chain constant region.

In some embodiments, the method further comprises introducing a sequence encoding a selectable marker.

As used herein, the term “selectable marker” or “selectable marker gene” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin (blast), and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonoacetyl-L-aspartate (PALA); human multi-drug resistance-1 (MDR-1; P-glycoprotein surface protein selectable by increased drug resistance or enriched by FACS); human CD25 (IL-2.alpha.; detectable by Mab-FITC); Methylguanine-DNA methyltransferase (MGMT; selectable by carmustine); rhamnose; and Cytidine deaminase (CD; selectable by Ara-C).

In further embodiments, provided herein is a method of making an antibody producing cell, comprising: providing a cell as described herein, introducing a sequence encoding an antibody heavy chain variable region to the upstream of the first sequence encoding antibody heavy chain constant regions 1, 2, and 3 on one strand of a genomic nucleic acid sequence of a cell, and introducing a sequence encoding an antibody light chain variable region to the upstream of the second sequence encoding antibody light chain constant region 1 on an opposite strand of the genomic nucleic acid sequence of the cell.

In some embodiments, provided herein is a method of making an antibody producing cell, further comprising using a single guide RNA to introduce both the sequences encoding an antibody heavy chain variable region on one strand of a genomic nucleic acid sequence of a cell and the sequence encoding an antibody light chain variable region on an opposite strand of the genomic nucleic acid sequence of the cell. In some embodiments, the method further comprises introducing a promoter sequence with the sequence encoding an antibody heavy chain variable region. In some embodiments, the method further comprises introducing a promoter sequence with the sequence encoding an antibody light chain variable region. In some embodiments, the promoter sequence is operably connected to the sequence encoding an antibody heavy chain variable region. In some embodiments, the promoter sequence is operably connected to the sequence encoding an antibody light chain variable region.

In some embodiments, the sequence encoding an antibody heavy chain variable region is operably connected to the first sequence encoding antibody heavy chain constant regions 1, 2, and 3. In some embodiments, the sequence encoding an antibody light chain variable region is operably connected to the second sequence encoding antibody light chain constant region 1.

In some embodiments, the method further comprises introducing guide RNAs through plasmid that expresses the guide RNA transiently.

As described herein, in embodiments, the editable cell is a eukaryotic cell. In some embodiments, the cell is a mammalian cell, a yeast cell, an insect cell, an algae cell, or a plant cell. In suitable embodiments, the cell is a Chinese hamster ovary (CHO) cell. Examples of CHO cells include, but are not limited to, CHO-K1, CHOK1SV, Potelligent CHOK1SV (FUT8-KO), CHO GS-KO, Xceed (CHOK1SV GS-KO), CHO-S, CHO DG44, CHO DXB11, CHOZN, or a CHO-derived cell. In some embodiments, the cell is a Chinese Hamster Ovary (CHO) cell.

In suitable embodiments, the cell is a human embryonic kidney (HEK) cell. In some embodiments, the cell is selected from the group consisting of HeLa, HEK293, H9, HepG2, MCF7, Jurkat, NIH3T3, PC12, PER.C6, BHK, VERO, SP2/0, NS0, YB2/0, EB66, C127, L cell, COS, e.g., COS1 and COS7, QC1-3, CHO-K1, CHOK1SV, Potelligent CHOK1SV (FUT8-KO), CHO GS-KO, Exceed (CHOK1SV GS-KO), CHO-S, CHO DG44, CHO DXB11, and CHOZN.

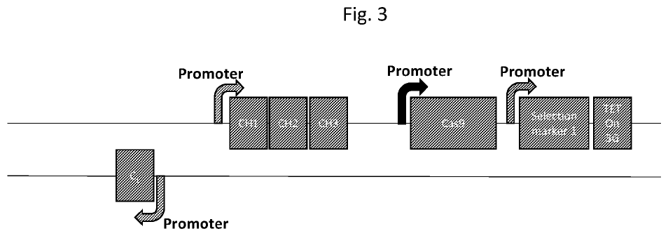

In further embodiments, provided herein is a cell, comprising a genomic nucleic acid sequence comprising, a first sequence encoding antibody heavy chain constant regions 1, 2, and 3 on one strand of the genomic nucleic acid sequence, wherein the first sequence is not flanked by a sequence encoding an antibody heavy chain variable region, and a second sequence encoding antibody light chain constant region 1 on an opposite strand of the genomic nucleic acid sequence, wherein the second sequence is not flanked by a sequence encoding an antibody light chain variable region. As described herein, suitably the first and the second sequences are on the opposite strands of the genomic nucleic acid within the same locus. See for Example, FIG. 3, illustrating an embodiment where the heavy chain and light chain regions are on the opposite strands of the genomic nucleic acid. In some embodiments, the first and the second sequences on the opposite strands within the same locus are separated by 1, 2, 3, 4, 5, 6, 7, 8, 9, or 10 nucleotide base pairs. In still further embodiments, the nucleic acids can be separated by a greater number of base pairs, and can even be on different chromosomes.

In some embodiments, the cell further comprises a first promoter operably connected to the first sequence and a second promoter operably connected to the second sequence. In some embodiments, the cell further comprises a bidirectional promoter operably connected to the first sequence and the second sequence on the opposite strands of the genomic nucleic acid in the same locus. In some embodiments, the first and the second sequences do not have a promoter connected to the sequence.

In some embodiments, the cell further comprises a sequence encoding a gene editing protein. In some embodiments, the gene editing protein is a CRISPR-associated gene editing protein. In suitable embodiments, the CRISPR-associated (Cas) nuclease is a Cas9 nuclease, or can be other Cas nucleases such as Cas12, Cas12i2, Cas13, Cas14, MAD7 (Cas12a), etc. In some embodiments, the Cas9 nuclease is a Cas9 nuclease that has reduced immunogenicity. In some embodiments, the gene editing protein is a zinc finger nuclease. In some embodiments, the gene editing protein is a TALENS. In some embodiments, the gene editing protein is a FokI nuclease. As described herein, the gene editing protein can be included as a part of the genomic sequence, or can be a separately provided sequence (e.g., via a vector) or as a RNP.

In some embodiments, the sequence encoding the gene editing protein is operably connected to an inducible promoter. By placing the gene editing protein under the control of an inducible promoter, the nuclease can be kept dormant or silent prior to its desired use as a gene editing tool. In some embodiments, the inducible promoter is a TET-on system, as described herein.

As described herein, the cell is suitably a eukaryotic cell. In some embodiments, the cell is a mammalian cell, a yeast cell, an insect cell, an algae cell, or a plant cell. In suitable embodiments, the cell is a Chinese hamster ovary (CHO) cell. Examples of CHO cells include, but are not limited to, CHO-K1, CHOK1SV, Potelligent CHOK1SV (FUT8-KO), CHO GS-KO, Xceed (CHOK1SV GS-KO), CHO-S, CHO DG44, CHO DXB11, CHOZN, or a CHO-derived cell. In some embodiments, the cell is a Chinese Hamster Ovary (CHO) cell. In suitable embodiments, the cell is a human embryonic kidney (HEK) cell. In some embodiments, the cell is selected from the group consisting of HeLa, HEK293, H9, HepG2, MCF7, Jurkat, NIH3T3, PC12, PER.C6, BHK, VERO, SP2/0, NS0, YB2/0, EB66, C127, L cell, COS, e.g., COS1 and COS7, QC1-3, CHO-K1, CHOK1SV, Potelligent CHOK1SV (FUT8-KO), CHO GS-KO, Exceed (CHOK1SV GS-KO), CHO-S, CHO DG44, CHO DXB11, and CHOZN.

In some embodiments, the cell further comprises a sequence encoding a selectable marker.

FIG. 3 shows an exemplary representation of a genomic sequence of the editable cell comprising the first and the second sequences on the opposite strands of the genomic nucleic acid located within the same locus. The genomic sequence also contains the sequence encoding the gene editing protein (Cas9), the sequence encoding the selection marker, and the sequence encoding the TET-on inducible promoter, which is provided as an exemplary embodiment.

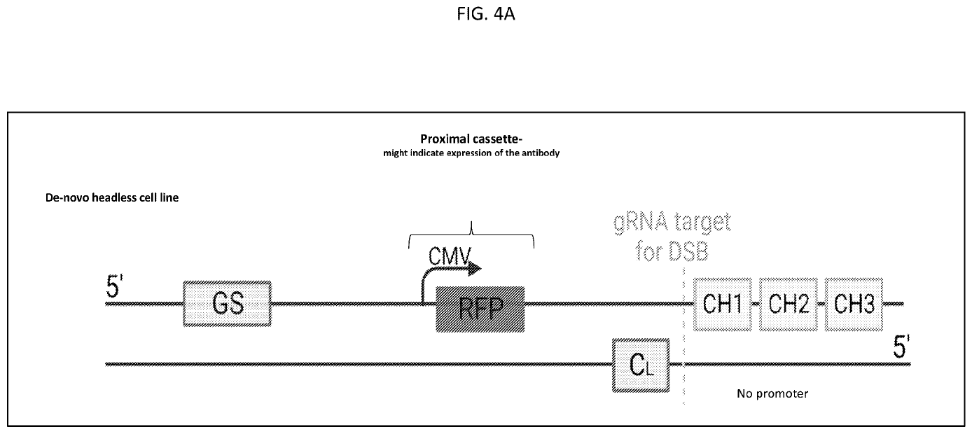

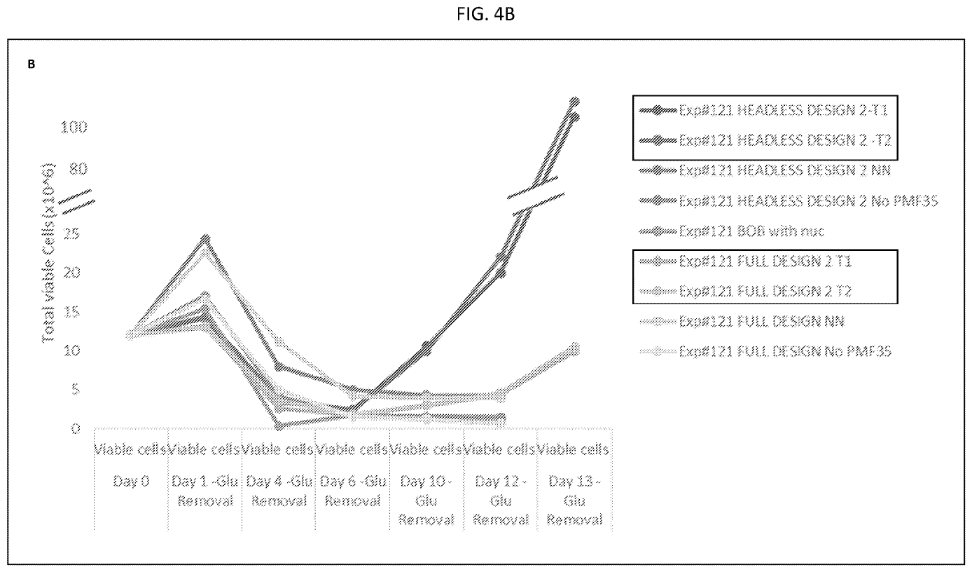

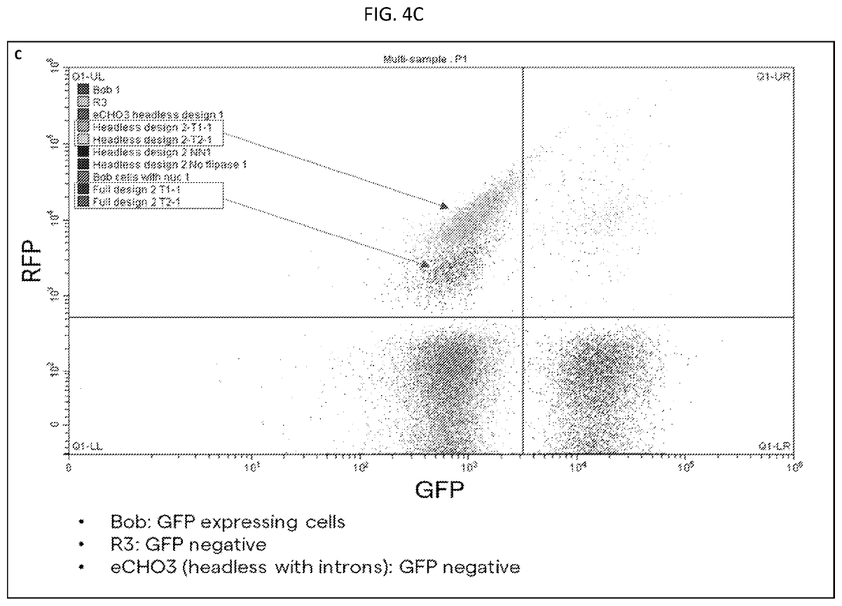

FIG. 4A shows a representation of a genomic sequence of a headless antibody that includes the heavy chain and light chain constant regions on the opposite strands of the genomic nucleic acid located within the same locus without promoters, so the headless antibody is not expressed. The genomic sequence, however, also encodes the selection marker (e.g., RFP) inserted using proximal cassette with CMV promoter that may be utilized to indicate expression of the antibody. FIG. 4B shows cells' recovery after transfection of a genomic sequence of a headless antibody as described herein, indicating successful generation and cloning of cells with genomic sequence of a headless antibody. FIG. 4C shows FACS analysis validating the successful integration of the cassette, as indicated by RFP expression compared to GFP expression in control cells.

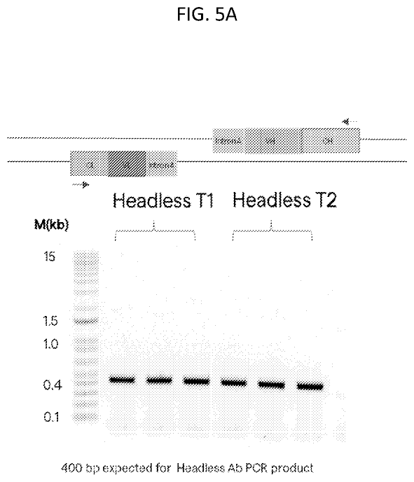

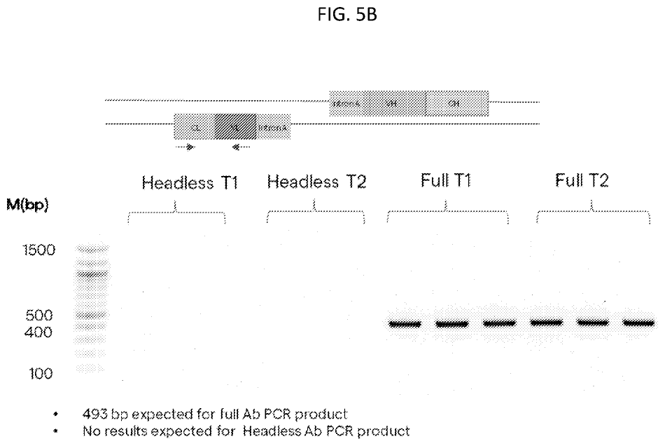

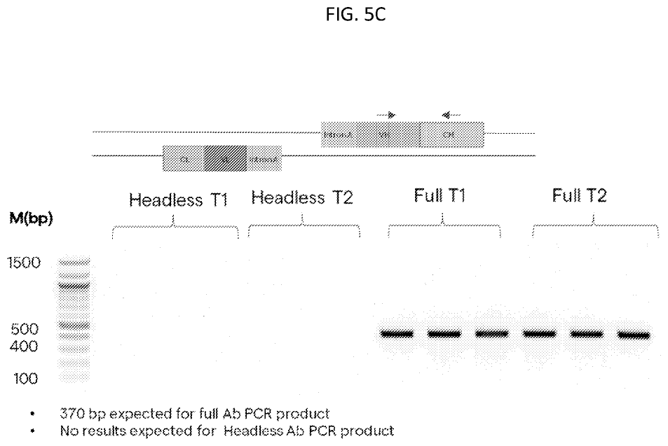

FIG. 5A shows the PCR result of a successfully generated and expressed headless antibody, which is indicated by the band at 400 bp. Therefore, as shown in FIG. 5B, a successful transfection of a non-expressing headless antibody does not show any bands with PCR, whereas PCR expression of the light chain region of the full antibody shows bands at 493 bp. Similarly, FIG. 5C shows bands at 370 bp for PCR expression of the heavy chain region of the full antibody, whereas no bands are shown for non-expressing headless antibody.

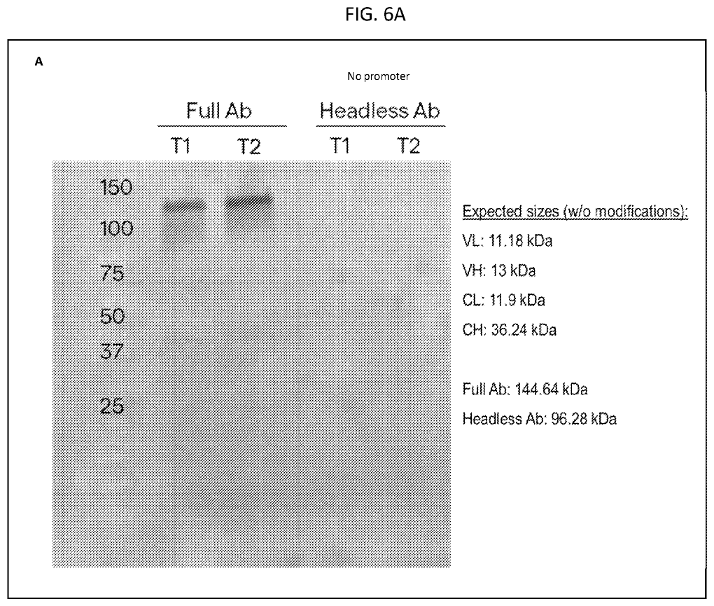

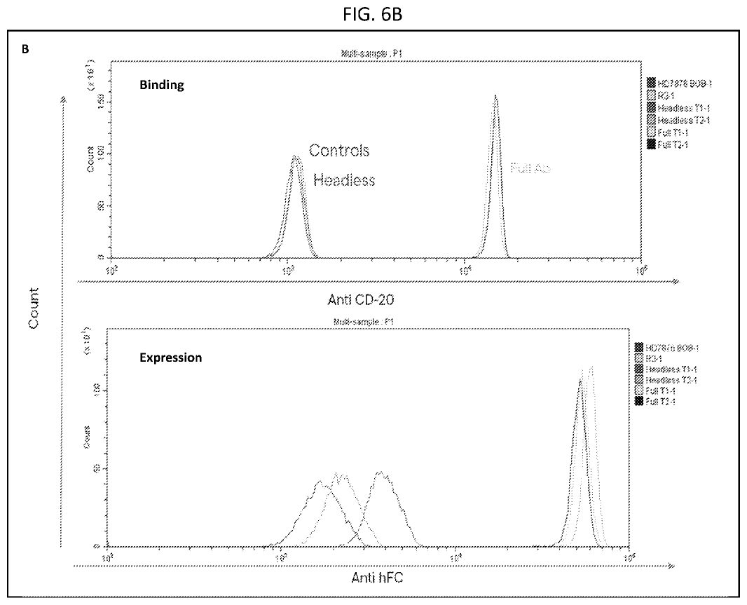

In FIG. 6A, the Western Blot result shows successful generation of non-expressing headless antibody. The expected size of a full antibody is 144.64 kDa and is clearly indicated by the bands in the left two columns whereas no bands are shown in the right two columns for headless antibody without promoters. FIG. 6B also shows successful generation of full and headless antibody through FACS analysis of functionality and expression of full and headless antibody. The result shows no binding or expression of headless antibody, similar to the control.

In some embodiments, provided herein is a method of making an antibody producing cell, further comprising introducing a promoter operably connected to the sequence encoding the antibody heavy chain region and a promoter operably connected to the sequence encoding the antibody light chain region. In some embodiments, the method further comprises introducing a promoter operably connected to the sequence encoding antibody.

In some embodiments, provided herein is a method of making an antibody producing cell, further comprising introducing a first sequence encoding a first selectable marker and a second sequence encoding a second selectable marker. In some embodiments, the method further comprises selecting a cell expressing the antibody using the first selectable marker. In some embodiments, the method further comprises selecting a cell expressing the antibody using the second selectable marker. In some embodiments, the method further comprises selecting a cell expressing the antibody using the first and the second selectable markers. In some embodiments, the method further comprises expressing the antibody in the cell.

Methods for expanding the cells produced using the methods described herein are known in the art, as are methods for recovering the antibodies produced thereby. Such methods, include for example, various column filtration methods, washing steps, as well as bead-based magnetic separation methods, etc.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

### EXAMPLES

### Example 1: Seamless Integration of Heavy Chain Variable Region and Light Chain Variable Region into Non-Expressing Headless IgG Antibody

### Method

Cells with non-expressing headless IgG antibody wherein one strand encodes the heavy chain constant region and the opposite strand encodes the light chain constant region are sub-cultured at a viable concentration of 0.3×10⁶ cells/mL into an appropriately sized Erlenmeyer shake-flask (CD-CHO medium). A guide RNA is selected and prepared for nucleofection. 100 μg of DNA template that consist of a VL domain that is encoded on one strand and the VH domain on the other strand are centrifuged briefly to collect contents and re-suspended with 25 μl of TE buffer to receive a concentration of 4 μg/μl.

Cas9 RNP complexes are prepared according to Table 1.

TABLE 1
RNP complexes

| | Cas9 | sgRNA | | DNA | TE | |
|---|---|---|---|---|---|---|
| Tube | protein | #9 | | template | buffer | |
| No. | (μl) | (μl) | | (μg, μl) | (μl) | Nucleofection |
| 1 | 3.2 | 3.2 | ssDNA | 0.4 μg (for 4 | 1.2 | + |
| | | | | samples), 2 μl | | |
| | | | | (out of | | |
| | | | | 0 2 μg/μl) | | |
| 2 | 3.2 | 3.2 | | 2 μg, 2 μl | 1.2 | + |
| | | | | (out of l μg/μl) | | |
| 3 | 3.2 | 3.2 | | 4 μg, 1 μl | 2.2 | + |
| 4 | 3.2 | 3.2 | | 12 μg, 3 μl | 0.2 | + |
| 5 | 3.2 | 3.2 | dsDNA | 0.4 μg (for 4 | 1.2 | + |
| | | | | samples), 2 μl | | |
| | | | | (out of | | |
| | | | | 0.2 μg/μl) | | |
| 6 | 3.2 | 3.2 | | 2 μg, 2 μl | 1.2 | + |
| | | | | (out of | | |
| | | | | 1 μg/μl) | | |
| 7 | 3.2 | 3.2 | | 4 μg, 1 μl | 2.2 | + |
| 8 | 3.2 | 3.2 | | 12 μg, 3 μl | 0.2 | + |
| 9 | 3.2 | 3.2 | − | − | 3.2 | + |
| 10 | 3.2 | − | ssDNA | 4 μg, 1 μł | 5.4 | + |
| 11 | − | 3.2 | | 4 μg, 1 μl | 5.4 | + |
| 12 | 3.2 | − | dsDNA | 4 μg, 1 μł | 5.4 | + |
| 13 | − | 3.2 | | 4 μg, 1 μł | 5.4 | + |
| 14 | − | − | | − | 9.6 | + |
| 15 | 3.2 | 3.2 | ssDNA | 4 μg, 1 μl | 2.2 | − |
| 16 | 3.2 | 3.2 | dsDNA | 4 μg, 1 μl | 2.2 | − |
| 17 | − | − | | − | 9.6 | − |

The complexes are incubated at room temperature for 30-60 minutes.

Cells with non-expressing headless IgG antibody are prepared for nucleofection by transferring 17×10⁶ cells to a new tube and centrifuged at 200 g for 5 min to remove as much media as possible. DNA templates according to Table 1 are added to the appropriate Cas9 RNP complexes as prepared. The cells are then re-suspended in 1196.8 μl of Nucleofection solution (1250 μl of SF solution prepared by mixing 1025 μl of SF solution with 225 μl of supplement 1). 70.4 μl of cells are added to each of the Cas9 RNP complex mixtures.

20 μl of the cells-nucleofection solution-RNP complex mixture are transferred to a cuvette in the nucleofection 16-well strip where cells are pulsed using the Lonza 4D nucleofector unit using program DU-158. The cells are left to sit in the nucleofection cuvettes for 10 min post nucleofection and added and mixed 80 μl of pre-warmed 10 ml of CD-CHO medium warmed to a 36.5° C. 100 μl of the final mixtures are transferred to the 12-well plate with 1.5 ml of a CD-CHO medium, kept in the incubator at 36.5° C. The cells are then incubated at 36.5° C., 5% CO₂ in a static incubator for 48 hours.

### Results

Integration efficiency assessment is performed using PCR, NGS, FACS analysis, and Western Blot analysis.

### Example 2: Seamless Integration of Heavy Chain Variable Region and Light Chain Variable Region into Non-Expressing Headless IgG Antibody

### Method

Cells with non-expressing headless IgG antibody wherein one strand encodes the heavy chain constant region and the opposite strand encodes the light chain constant region are sub-cultured at a viable concentration of 0.3×10⁶ cells/mL into an appropriately sized Erlenmeyer shake-flask (CD-CHO medium). A guide RNA (sgRNA #30) with the following sequence (5′ to 3′): ccacggtccgcttCCATGCA (SEQ ID NO:1) is selected and prepared for nucleofection. 100 μg of DNA template that consist of a VL domain that is encoded on one strand and the VH domain on the other strand are centrifuged briefly to collect contents and re-suspended with 19.3 μl of TE buffer to receive a concentration of 5.2 μg/μl.

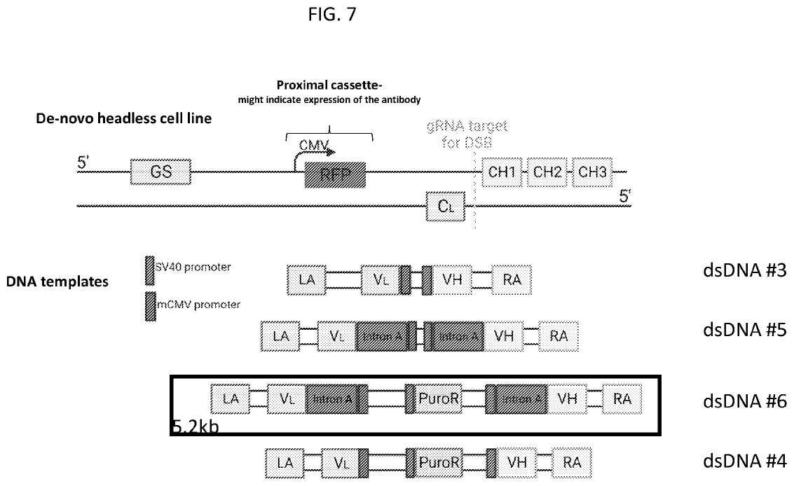

FIG. 7 shows schematic representation of various DNA templates for integrating both heavy chain and light chain variable regions to the genomic sequence of a headless antibody wherein the heavy chain and light chain constant regions are on the opposite strands of the genomic nucleic acid. The DNA templates contain Puromycin sequence for antibiotic selection and promoter sequences connected to heavy and light chain variable region sequences. dsDNA #6 is selected as the DNA template for preparing Cas9 RNP complexes for transfection.

Cas9 RNP complexes are prepared according to Table 2.

TABLE 2
RNP complexes

| | Cas9 | sgRNA | | DNA | TE | |
|---|---|---|---|---|---|---|
| Tube | protein | #30 | | template | buffer | |
| No. | (μl) | (μl) | | (μg, μl | (μl) | Nucleofection |
| 1 | 3.2 | 3.2 | Puro + | 5.2 μg, 2 μl | 1.2 | + |
| | | | introns | (out of | | |
| | | | dsDNA | 2 6 μg/μl) | | |
| | | | #6 | Dilote 1:2) | | |
| 2 | 3.2 | 3.2 | 5265 bp | 10.4 μg, 2 μl | 1.2 | + |
| 3 | 3.2 | − | | 10.4 μg, 2 μl | 4.4 | + |
| 4 | − | 3.2 | | 10.4 μg, 2 μl | 4.4 | + |
| 5 | − | − | | − | 9.6 | + |
| 6 | 3.2 | 3.2 | | 10.4 μg, 2 μl | 1.2 | [symbol] |
| 7 | − | − | | − | 9.6 | [symbol] |

The complexes are incubated at room temperature for 30-60 minutes.

Cells with non-expressing headless IgG antibody are prepared for nucleofection by transferring 8×10⁶ cells to a new tube and centrifuged at 200 g for 5 min to remove as much media as possible. DNA templates according to Table 2 are added to the appropriate Cas9 RNP complexes as prepared. The cells are then re-suspended in 563.2 μl of Nucleofection solution (492 μl of SF solution with 108 μl of supplement 1). 70.4 μl of cells are added to each of the Cas9 RNP complex mixtures.

20 μl of the cells-nucleofection solution-RNP complex mixture are transferred to a cuvette in the nucleofection 16-well strip where cells are pulsed using the Lonza 4D nucleofector unit using program DU-158. The cells are left to sit in the nucleofection cuvettes for 10 min post nucleofection and added and mixed 80 μl of pre-warmed 10 ml of CD-CHO medium warmed to a 36.5° C. 100 μl of the final mixtures are transferred to the 12-well plate with 1.5 ml of a CD-CHO medium, kept in the incubator at 36.5° C. The cells are then incubated at 36.5° C., 5% CO₂ in a static incubator for 24 hours.

24 hours after, the medium was replaced with CD-CHO+6 ug/mL of Puromycin. Cells were grown in static incubator until negative control were died. Cell viability assessment was performed using Vi-Cell device.

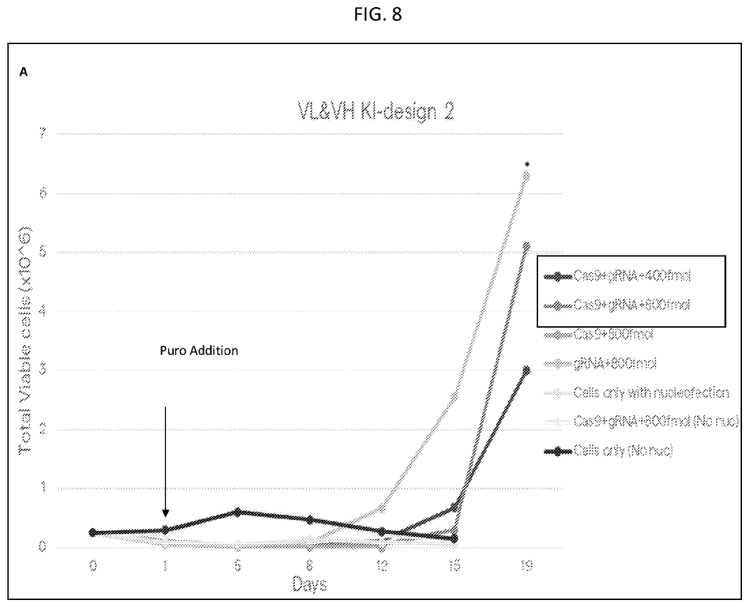

FIG. 8 shows cells' recovery following Puromycin antibiotic selection of cells transfected with heavy chain and light chain variable regions. As indicated by the *, negative control of cells with DNA template and gRNA recovered, with the two far right lines indicating recovery of cells transfected with heavy and light chain variable region using DNA template as described herein.

### Results

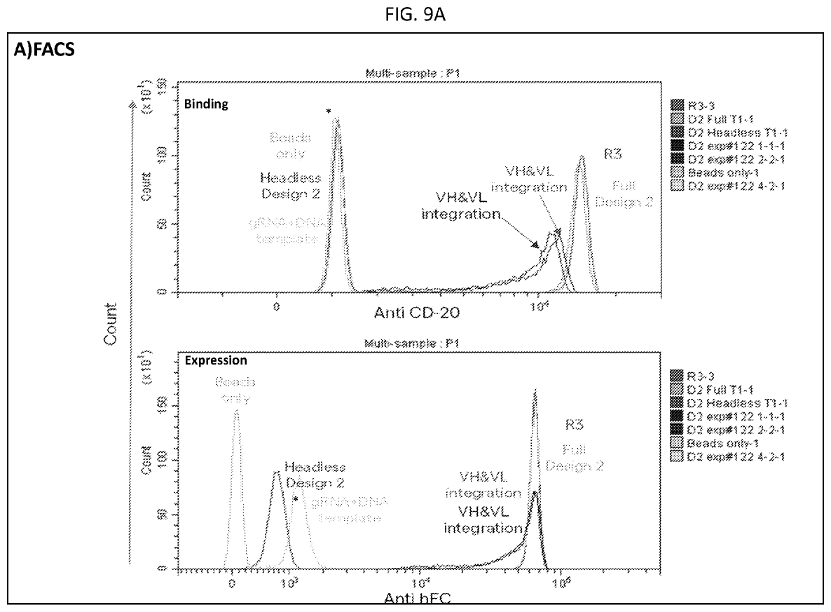

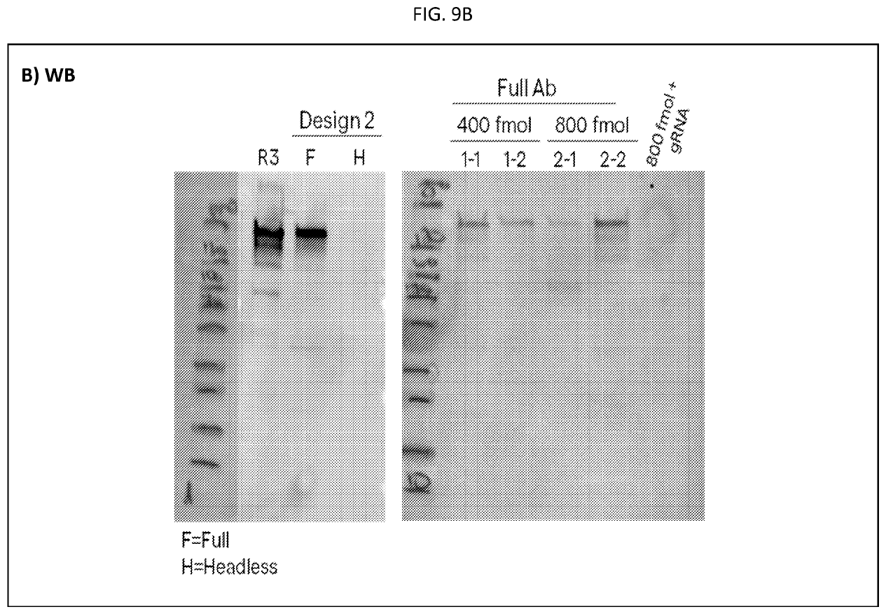

FACS analysis as shown in FIG. 9A shows successful generation of full antibody through seamless integration of heavy and light chain variable regions into headless cell line. FIG. 9B shows antibody expression as validated by WB, also validating successful generation of full antibody through seamless integration of heavy and light chain variable regions into headless cell line. As expected, non-expressing headless antibody did not show any binding or expression whereas successful integration of the heavy and light chain variable regions with promoters generated full antibody.

dsDNA #6 sequence (5′ to 3′);
(SEQ ID NO: 2)
tgtgcttctcgtagtcggccttggacagggtcagggtggaggacaggctgtaggtgctgtccttggagtcctgctcggtgacggattcctgg
gagttgccggactgcagggcgttgtccaccttccactgcaccttggcctcgcgggggtagaagttgttcagcaggcagaccacgctggcg
gtgccggacttcagctgctcgtcggagggtgggaagatgaacacggagggagcggccacggtccgcttgatttccagcttggtgcctccg
ccgaaggtgggggggttggaggtccactgctggcagtagtaggtggcggcgtcctcggcttccacccgggagatggtcagggagtagg
aggtgccagagccagagccggagaaccgcacgggcacgccagaggccaggttggaggtggcgtagatccagggcttggggctggag
ccgggcttctgctggaaccagtggatgtaggacacggaggaggaggcccggcaggtcattgtcactttctcgccagggctggcggacag
gatggcaggggactgggacagcacgatctggccccgagacatgatcacggaggcggagatcagcagaaaggagatgatctgcacctg
gaagtccatggtggcggcgacttcagaaaagacccatggaaaggaacagtctgttagtctgtcagctattatgtctggtggcgcgcgcggc
agcaacgagtactgctcagactacactgccctccaccgttaacagcaccgcaacgggagttacctctgactcttatcagaacacaacaactc
agctgcctgcatcttcttctgccgctgccttaagtcttccaaatgcgtcageggtgcaagcccgctccccgagctcattttcagacacataccc
taccgccacggccttgtgcggcacactggtggtggtgggcatcgtgctgtgcctaagtctggcctccactgttaggagcaaggagctgccg
agcgaccatgagtcgctggaggcatgggagcagggctcggatgtagaagctccgccgctaccggagaagagcccatgtccggaacac
gtacccgagattcgcgtggagatcccacgttatgtttaataaaaactgcgggcactggggacggtggtgttgtatatgtgaatttgtaaataat
aaatgagaccccatcctgtaaaaatacagagtccgtgtcagtctctgaaggacagtgtattggcatatagccaataaagagagttgtggcaa
agagccatgttatggattagtaatggaaagtatcgtcaccaataggggagtggtcaataatggtcaataacccacacctataggctaagctat
accatcacctataacatgaggaagcgggggtgtatagaccccaagccaaaaacagtatagcatgcataagaagccaagggggtgggcct
atagactctataggcggtacttacgtcactcttggcacggggaatccgcgttccaatgcaccgttcccggccgcggaggctggateggtcc
cggtgtcttctatggaggtcaaaacagcgtggatggcgtctccaggcgatctgacggtaccgacgctggtcgcgcctcttatacccacgtag
aacgcagctcagccaatagaatgcgtgccaatatggaatttccaggggaaaaccggggcggtgttacgttttggctgccctttcacttcccat
tgacgtgtattggctcgagaacggtactttcccattaatcagctatgggaaagtaccgtttaaaggtcacgttgcattagtttcaatagcccattg
acgtcaatggtgggaaagtacatggcgttttaattaaattggctggaaaaacccaatgactcacccctattgaccttatgtacgtgccaataatg
ggaaaaacccattgactcaccccctattgaccttttgtactgggcaaaacccaatggaaagtccctattgactcagtgtacttggctccaatgg
gactttcctgttgattcacccctattgaccttatgtactgggcaaaacccattggaaagtccctaatgactcagtaccagAACGatgattaga
cttagtactgccttttcctagtactcccccccgccagactgaggcggaaagaaccagctgtggaatgtgtgtcagttagggtgtggaaagtcc
ccaggctccccagcaggcagaagtatgcaaagcatgcatctcaattagtcagcaaccaggtgtggaaagtccccaggctccccagcagg
cagaagtatgcaaagcatgcatctcaattagtcagcaaccatagtcccgcccctaactccgcccatcccgcccctaactccgcccagttccg
cccattctccgccccatggctgactaattttttttatttatgcagaggccgaggccgcctcggcctctgagctattccagaagtagtgaggagg
cttttttggaggcctaggcttttgcaaaCGatgaccgagtacaagcccacggtgcgcctcgccacccgcgacgacgtcccccgggccgta
cgcaccctegccgccgcgttcgccgactaccccgccacgcgccacacegtcgacccggaccgccacatcgagcgggtcaccgagctg
caagaactcttcctcacgcgcgtcgggctcgacatcggcaaggtgtgggtcgcggacgacggcgccgcggtggcggtctggaccacgc
cggagagcgtcgaagcgggggcggtgttcgccgagatcggcccgcgcatggccgagttgagcggttcccggctggccgcgcagcaac
agatggaaggcctcctggcgccgcaccggcccaaggagcccgcgtggttcctggccaccgtcggcgtctcgcccgaccaccagggca
agggtctgggcagcgccgtcgtgctccccggagtggaggcggccgagcgcgccggggtgcccgccttcctggagacctccgegcccc
gcaacctccccttctacgagcggctcggcttcaccgtcaccgccgacgtcgaggtgcccgaaggaccgcgcacctggtgcatgacccgc
aagcccggttaagcgggatccaccggatctagtaagatacattgatgagtttggacaaaccacaactagaatgcagtgaaaaaaatgctttat
ttgtgaaatttgtgatgctattgctttatttgtaaccattataagctgcaataaacaagttatgattagacttagtactgccttttcctagtactccccc
ccgccagactgaggcggaaagaaccagctgtggaatgtgtgtcagttagggtgtggaaagtccccaggctccccagcaggcagaagtat
gcaaagcatgcatctcaattagtcagcaaccaggtgtggaaagtccccaggctccccagcaggcagaagtatgcaaagcatgcatctcaat
tagtcagcaaccatagtcccgcccctaactccgcccatcccgcccctaactccgcccagttccgcccattctccgccccatggctgactaatt
ttttttatttatgcagaggccgaggccgcctcggcctctgagctattccagaagtagtgaggaggcttttttggaggcctaggcttttgcaaaat
cagatcgcctggagacgccatccacgctgttttgacctccatagaagacaccgggaccgatccagcctccgcggccgggaacggtgcatt
ggaacgcggattccccgtgccaagagtgacgtaagtaccgcctatagagtctataggcccacccccttggcttcttatgcatgctatactgttt
ttggcttggggtctatacacccccgcttcctcatgttataggtgatggtatagcttagcctataggtgtgggttattgaccattattgaccactccc
ctattggtgacgatactttccattactaatccataacatggctctttgccacaactctctttattggctatatgccaatacactgtccttcagagact
gacacggactctgtatttttacaggatggggtctcatttattatttacaaattcacatatacaacaccaccgtccccagtgcccgcagtttttatta
aacataacgtgggatctccacgcgaatctcgggtacgtgttccggacatgggctcttctccggtagcggcggagcttctacatccgagccct
gctcccatgcctccagcgactcatggtcgctcggcagctccttgctcctaacagtggaggccagacttaggcacagcacgatgcccaccac
caccagtgtgccgcacaaggccgtggcggtagggtatgtgtctgaaaatgagctcggggagcgggcttgcaccgctgacgcatttggaa
gacttaaggcagcggcagaagaagatgcaggcagctgagttgttgtgttctgataagagtcagaggtaactcccgttgcggtgctgttaacg
gtggagggcagtgtagtctgagcagtactcgttgctgccgcgcgcgccaccagacataatagctgacagactaacagactgttcctttccat
gggtcttttctgaagatcggcggtggatgggctggtccctgatcctgctgttcctggtcgccgtggccacccgggtgctgtctcaggtgcag
ctgcagcagcctggcgccgagctggtcaaacctggegcctccgtgaagatgtcctgcaaggcctccggctacaccttcaccagctacaac
atgcactgggtcaaacagacccccggcagaggcctggaatggatcggcgccatctaccccggcaacggcgacacctcctacaaccaga
agttcaagggcaaggccaccctgaccgccgacaagtcctcctccaccgcctacatgcagctgtcctccctgacctccgaggactccgccg
tgtactactgcgcccggtccacctactacggcggcgactggtacttcaacgtgtggggcgctggcaccaccgtgaccgtgtcttctgcttcc
accaagggcccctccgtgttccctctggccccctccagcaagtccacctctggcggcaccgctgccctgggctgcctggtcaaagactact
tccccgagcccgtgacagtgtcctggaactctggcgccctgaccagcggcgtgcacaccttccctgccgtgctgcagtcctccggcctgta
ctccctgtccagcgtggtcacagtgccctcctccagcctgggcacccagacetaca

What is claimed is:

1. A cell, comprising a genomic nucleic acid sequence comprising,
a first sequence encoding antibody heavy chain constant regions 1, 2 and 3 on one strand of the genomic nucleic acid sequence, wherein the first sequence is not flanked by a sequence encoding an antibody heavy chain variable region, and
a second sequence encoding antibody light chain constant region 1 on an opposite strand of the genomic nucleic acid sequence, wherein the second sequence is not flanked by a sequence encoding an antibody light chain variable region.

2. The cell of claim 1, further comprising a sequence encoding a selectable marker.

3. The cell of claim 1 or 2, further comprising a sequence encoding a gene editing protein.

4. The cell of claim 3, wherein the gene editing protein is a Cas gene editing protein.

5. The cell of claim 3, wherein the gene editing protein is selected from Cas9, Cas12, Cas12i2 TALENS, MAD7 nuclease and a Zinc Finger Nuclease.

6. The cell of claim 5, wherein the gene editing protein is Cas9.

7. The cell of any one of claims 1-6, wherein the gene editing protein sequence is operably connected to an inducible promoter.

8. The cell of claim 7, wherein the inducible promoter is a TET-on system.

9. The cell of any one of claims 1-8, further comprising a first promoter operably connected to the first sequence and a second promoter operably connected to the second sequence.

10. The cell of any one of claims 1-9, wherein the cell is a high expressing, stable clone.

11. The cell of claim 10, wherein the cell is a Chinese Hamster Ovary (CHO) cell.

12. A method of producing an editable cell, comprising:
introducing a first sequence encoding antibody heavy chain constant regions 1, 2 and 3 on one strand of a genomic nucleic acid sequence of a cell; and
introducing a second sequence encoding antibody light chain constant region 1 on an opposite strand of the genomic nucleic acid sequence of the cell.

13. The method of claim 12, further comprising introducing a sequence encoding a selectable marker.

14. The method of claim 12 or 13, further comprising introducing a sequence encoding a gene editing protein to the genomic nucleic acid sequence.

15. The method of claim 14, wherein the gene editing protein is a Cas gene editing protein.

16. The method of claim 14, wherein the gene editing protein is selected from Cas9, Cas12, Cas12i2, TALENS, MAD7 nuclease and a Zinc Finger Nuclease.

17. The method of claim 16, wherein the gene editing protein is Cas9.

18. The method of any one of claims 12-17, wherein the gene editing protein sequence is operably connected to an inducible promoter.

19. The method of claim 18, wherein the inducible promoter is a TET-on system

20. The method of any one of claims 12-19, further comprising introducing a first promoter operably connected to the first sequence and a second promoter operably connected to the second sequence.

21. The method of any one of claims 12-20 wherein the cell is a high expressing, stable clone.

22. The method of claim 21, wherein the cell is a Chinese Hamster Ovary (CHO) cell.

23. A method of making an antibody producing cell, comprising:
providing a cell of any one of claims 1-11;
introducing a sequence encoding an antibody heavy chain variable region to the first sequence upstream of the antibody heavy chain constant regions; and
introducing a sequence encoding an antibody light chain variable region to the second sequence upstream of the antibody light chain constant region 1.

24. The method of claim 23, further comprising introducing a first promoter operably connected to the sequence encoding the antibody heavy chain variable region and a second promoter operably connected to the sequence encoding the antibody light chain variable region.

25. The method of claim 23 or 24 further comprising expressing the antibody in the cell.

26. The method of any one of claims 23-25, further comprising introducing a sequence encoding a selectable marker.

27. The method of claim 26, further comprising selecting a cell expressing the antibody using the selectable marker.

28. A method of making an antibody producing cell, comprising:
using a single guide RNA to introduce both a sequence encoding an antibody heavy chain variable region to a first sequence upstream of an antibody heavy chain constant region on one strand of a genomic nucleic acid sequence of a cell and a sequence encoding an antibody light chain variable region to a second sequence upstream of an antibody light chain constant region 1 on an opposite strand of the genomic nucleic acid sequence of the cell.

29. The method of claim 28, further comprising introducing a first promoter with the sequence encoding the antibody heavy chain variable region.

30. The method of claim 29, wherein the first promoter is operably connected to the sequence encoding the antibody heavy chain variable region.

31. The method of any one of claims 28-30, further comprising introducing a second promoter with the sequence encoding the antibody light chain variable region.

32. The method of claim 31, wherein the second promoter is operably connected to the sequence encoding the antibody light chain variable region.


SEQUENCE LISTING (2 sequences)

SEQ ID NO: 1 - DNA, 20 bp, synthetic construct
  misc_feature 1..20: note = Synthetic: guide RNA
  source 1..20: mol_type = other DNA
ccacggtccgcttccatgca

SEQ ID NO: 2 - DNA, 5265 bp, synthetic construct
  misc_feature 1..5265: note = Synthetic: dsDNA #6 sequence
  source 1..5265: mol_type = other DNA
tgtgcttctcgtagtcggccttggacagggtcagggtggaggacaggctgtaggtgctgtccttggagtcctgctcggtgacggattcctgggagttgccggactgcagggcgttgtccaccttccactgcaccttggcctcgcgggggtagaagttgttcagcaggcagaccacgctggcggtgccggacttcagctgctcgtcggagggtgggaagatgaacacggagggagcggccacggtccgcttgatttccagcttggtgcctccgccgaaggtgggggggttggaggtccactgctggcagtagtaggtggcggcgtcctcggcttccacccgggagatggtcagggagtaggaggtgccagagccagagccggagaaccgcacgggcacgccagaggccaggttggaggtggcgtagatccagggcttggggctggagccgggcttctgctggaaccagtggatgtaggacacggaggaggaggcccggcaggtcattgtcactttctcgccagggctggcggacaggatggcaggggactgggacagcacgatctggccccgagacatgatcacggaggcggagatcagcagaaaggagatgatctgcacctggaagtccatggtggcggcgacttcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaacacaacaactcagctgcctgcatcttcttctgccgctgccttaagtcttccaaatgcgtcagcggtgcaagcccgctccccgagctcattttcagacacataccctaccgccacggccttgtgcggcacactggtggtggtgggcatcgtgctgtgcctaagtctggcctccactgttaggagcaaggagctgccgagcgaccatgagtcgctggaggcatgggagcagggctcggatgtagaagctccgccgctaccggagaagagcccatgtccggaacacgtacccgagattcgcgtggagatcccacgttatgtttaataaaaactgcgggcactggggacggtggtgttgtatatgtgaatttgtaaataataaatgagaccccatcctgtaaaaatacagagtccgtgtcagtctctgaaggacagtgtattggcatatagccaataaagagagttgtggcaaagagccatgttatggattagtaatggaaagtatcgtcaccaataggggagtggtcaataatggtcaataacccacacctataggctaagctataccatcacctataacatgaggaagcgggggtgtatagaccccaagccaaaaacagtatagcatgcataagaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggtaccgacgctggtcgcgcctcttatacccacgtagaacgcagctcagccaatagaatgcgtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtaccagaacgatgattagacttagtactgccttttcctagtactcccccccgccagactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaacgatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggttaagcgggatccaccggatctagtaagatacattgatgagtttggacaaaccacaactagaatgcagtgaaaaaaatgctttatttgtgaaatttgtgatgctattgctttatttgtaaccattataagctgcaataaacaagttatgattagacttagtactgccttttcctagtactcccccccgccagactgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaatcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgaagatcggcggtggatgggctggtccctgatcctgctgttcctggtcgccgtggccacccgggtgctgtctcaggtgcagctgcagcagcctggcgccgagctggtcaaacctggcgcctccgtgaagatgtcctgcaaggcctccggctacaccttcaccagctacaacatgcactgggtcaaacagacccccggcagaggcctggaatggatcggcgccatctaccccggcaacggcgacacctcctacaaccagaagttcaagggcaaggccaccctgaccgccgacaagtcctcctccaccgcctacatgcagctgtcctccctgacctccgaggactccgccgtgtactactgcgcccggtccacctactacggcggcgactggtacttcaacgtgtggggcgctggcaccaccgtgaccgtgtcttctgcttccaccaagggcccctccgtgttccctctggccccctccagcaagtccacctctggcggcaccgctgccctgggctgcctggtcaaagactacttccccgagcccgtgacagtgtcctggaactctggcgccctgaccagcggcgtgcacaccttccctgccgtgctgcagtcctccggcctgtactccctgtccagcgtggtcacagtgccctcctccagcctgggcacccagacctaca